(19) United States
(12) Patent Application Publication
(10) Pub. No.: US 2004/0115660 A1
(43) Pub. Date: Jun. 17, 2004

(54) Method of diagnosising abnormalities by screening for tyrosine kinase like molecules or their signalling mediators

(76) Inventors: Steven Stacker, Victoria (AU); Michael M Halford, Victoria (AU); Andrew W Wilks, Victoria (AU); Michael Buchert, Montpellier (FR); Christopher Hovens, Victoria (AU)

Correspondence Address: Paul J Esatto Jr, Scully Scott Murphy & Presser, 400 Garden City Plaza, Garden City, NY, 11530, US

(21) Appl. No.: 10/343,103

(22) PCT Filed: Jul. 27, 2001
(86) PCT No.: PCT/AU01/00932

(30) Foreign Application Priority Data
Jul. 28, 2000 (AU) PQ9091

(51) Int. Cl. C12Q 1/68

(52) U.S. Cl. 435/6

(57) ABSTRACT

The present invention relates generally to a method for detecting abnormalities or a propensity for an abnormality to occur in an animal such as a human. The abnormality of the present invention relates generally to conditions arising from interruption in signalling associated with receptor-type tyrosine kinase-like molecules and in particular RYK (related to tyrosine kinase) receptors. Such abnormalities include aberrations in the normal morphogenesis of craniofacial structures including secondary palate as well as a range of aberrations resulting in neural conditions affecting angiogenesis and conditions affecting muscle development or maintenance. The present invention permits the early diagnosis of such defects and conditions such as in utero and provides a means for genetic or other therapeutic intervention.

### FIELD OF THE INVENTION

The present invention relates generally to a method for detecting abnormalities or a propensity for an abnormality to occur in an animal such as a human. The abnormality of the present invention relates generally to conditions arising from interruption in signalling associated with receptor-type tyrosine kinase-like molecules and in particular RYK (related to tyrosine kinase) receptors. Such abnormalities include aberrations in the normal morphogenesis of craniofacial structures including secondary palate as well as a range of aberrations resulting in neural conditions affecting angiogenesis and conditions affecting muscle development or maintenance. The present invention permits the early diagnosis of such defects and conditions such as in utero and provides a means for genetic or other therapeutic intervention.

### BACKGROUND OF THE INVENTION

Bibliographic details of the publications numerically referred to in this specification are collected at the end of the description.

The reference to any prior art in this specification is not, and should not be taken as, an acknowledgment or any form of suggestion that that prior art forms part of the common general knowledge in Australia and any other country.

The phosphorylation of tyrosine residues on protein substrates is a pathway whereby signals of growth and differentiation are transmitted by growth factor receptors and transforming oncogenes. Evidence for this role of tyrosine phosphorylation came from the identification of receptors which bind known soluble growth factors. For example, the receptors for epidermal growth factor (EGF), platelet derived growth factor (PDGF) and colony stimulating factor-1 (CSF-1) were all shown to be transmembrane molecules with the cytoplasmic regions defining a tyrosine kinase catalytic domain.

The other line of evidence for a critical role played by tyrosine phosphorylation in growth control came from the study of viral oncogenes. These genes were shown to be directly involved in growth dysregulation by observations of a change in cell growth following introduction of DNA encoding these genes into fibroblasts. All oncogenes have been shown to have close cellular homologues (proto-oncogenes). One of the first identified oncogenes was v-src, the cellular homologue (c-src) is the prototypical representative of the family of cytoplasmic tyrosine kinases which, following myristylation, become associated with the inner leaf of the cell membrane.

Protein-tyrosine kinases (PTKs) represent a family of phosphotransferases related by their conserved catalytic domains. Phylogenetic analysis of this family suggests that several subfamilies of the PTKs exist based on the organization of their non-catalytic sequences. These families include (i) the Src related PTKs such as c-yes, c-lyn and hck; (ii) the JAK family; and (iii) a number of subfamilies of growth factor receptors.

In particular, these previously known PTKs contain the Rossman motif which is putatively associated with ATP binding. The Rossman motif has three invariant glycine residues in a six amino acid cluster as follows: Gly-X-Gly-X-X-Gly, where X is an amino acid residue.

Proteins having receptor-like PTK-like properties have been discovered representing a new family of proteins related to receptor-type PTKs but exhibiting one or more of the following characteristics: an altered Rossman motif, a unique tri-amino acid sequence in the kinase catalytic domain and/or an extracellular region comprising leucine-rich regions. The proteins having the receptor-type PTK-like properties were designated herein “RYK” for “related to tyrosine kinases” (4-6) and are also described in International Patent Application No. PCT/AU93/00210 [WO 93/23429] which is incorporated herein by reference.

Secondary palate formation is a complex process which is frequently disturbed in mammals, resulting in the birth defect cleft palate (1, 2). Gene targeting has identified components of cytokine/growth factor signalling systems such as TGF-α/EGFR, Eph receptors B2 and B3, TGF-β2, TGF-β3 and activin-βA (3) as important regulators of secondary palate development. In accordance with the present invention, the inventors have demonstrated that the mouse orphan receptor RYK is essential for normal development and morphogenesis of craniofacial structures including the secondary palate. Furthermore, biochemical data implicate RYK in signalling mediated by Eph receptors and the cell junction-associated AF-6 (Afadin) indicating the importance of signal crosstalk between members of different RTK subfamilies.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc. A sequence listing is provided after the claims.

An aspect of the present invention contemplates a method for detecting a likelihood for progression of a developmental abnormality in an animal or for diagnosing the genetic or biochemical basis behind a particular developmental abnormality in said animal, said method comprising screening for the presence of a functional RYK molecule or its homologue or a mediator of RYK signalling or a functional nucleic acid molecule encoding said RYK or its signalling mediator or a transcript thereof wherein the absence of a functional RYK or its homologue or its signalling mediator or a mutation in the nucleic acid molecule encoding RYK or its signalling mediator or the presence of an aberration in the RYK transcript or signalling mediator transcript is indicative of or the likelihood of progression to a developmental disorder in said animal.

Another aspect of the present invention provides a method for detecting a likelihood for the progression of abnormal craniofacial structures in an animal or for diagnosing the genetic or biochemical basis behind a particular craniofacial abnormality or for detecting neurological conditions, conditions affecting angiogenesis and/or muscle development or maintenance in said animal, said method comprising screening for the presence of a functional RYK molecule or its homologue or a mediator of RYK signalling or a functional nucleic acid molecule encoding said RYK or its signalling mediator or a transcript thereof wherein the absence of a functional RYK or its homologue or its signalling mediator or a mutation in the nucleic acid molecule encoding RYK or its signalling mediator or the presence of an aberration in the RYK transcript or signalling mediator transcript is indicative of or the likelihood of progression to an abnormality in said animal.

Still another aspect of the present invention provides a method for detecting an abnormal genomic coding sequence for a RYK or a protein having RYK-like properties in an animal subject, said method comprising contacting a genetic sample from said subject with one or more oligonucleotide primers specific for a part of the naturally occurring genomic sequence for said protein or for an abnormal coding sequence for said protein for a time and under conditions sufficient for said oligonucleotides to hybridize to said genomic sequence and then screening for said hybridization.

### BRIEF DESCRIPTION OF THE DRAWINGS

FIG. 1 is representation of the creation of a null allele of Ryk. (a) Gene targeting strategy applied to the mouse Ryk locus (drawn to scale). The homologous recombination event deleted 14.5 kb of genomic DNA including exons (numbered boxes) encoding >95% of the RYK extracellular domain and the entire transmembrane (TM) domain. This was replaced with a promoterless IRES.βgeo.pA cassette which, using the endogenous exon 2 splice acceptor site, was expected to faithfully report the activity of the Ryk promoter. Stop codons were introduced into all three reading frames via fusion with the IRES. The location of probes used for Southern blotting analysis of the targeted allele, the sizes of expected hybridization fragments and the PCR primers used for genotyping (GTwt, GTR and GTKO3; not to scale) are indicated. Only relevant restriction sites are shown. IRES, picornavirus internal ribosome entry site; LRM, leucine-rich motif; TBC, putative tetrabasic protease cleavage site; βgeo, β-Gal-neo fusion gene; pA, SV40 polyadenylation signal; pBS II KS(−), pBluescript II plasmid (Stratagene); H, HindIII; S, SacI; X, AbaI; E, EcoRI; A, ApaI; K, KpnI; N, NotI. (b) Southern blotting analysis of two targeted ES cell clones (RKO.44 and RKO 2.16) with the 5′ flanking probe, βgeo-specific probe and 3′ flanking probe demonstrating single site-specific integration of the targeting vector by homologous recombination. Sizes of fragments derived from the wild-type and targeted Ryk alleles are indicated. (c) Germline transmission of the targeted Ryk allele from cell line RKO.44. Two allele, three primer PCR was performed on tail DNA (or ES cell DNA from RKO.44, right lane) isolated from progeny of Ryk^{+/-} intercrosses. The size of DNA standards (in bp) is indicated. targ, PCR product diagnostic of targeted Ryk allele; wt, PCR product diagnostic of wild-type Ryk allele. (d) Multiplex RT-PCR analysis of total RNA isolated from 18.5 dpc embryos of the indicated genotypes. RT⁺, cDNA synthesis reactions containing reverse transcriptase; RT-, cDNA synthesis reactions lacking reverse transcriptase; e, PCR lacking template cDNA; IC, intracellular; EC, extracellular. The size of DNA standards (in bp) is indicated. (e) Blotting analysis of reduced anti-RYK immunoprecipitates from neonatal mouse kidney lysates with a GST.AF-6.PDZ affinity reagent. The structure of proteolytically processed RYK and the reactivity of reagents used to analyse expression of the receptor is depicted at left. The positions of the β subunit (apparent molecular weight 50 kD), which results from proteolytic cleavage of the RYK extracellular domain at or near the TBC site (5) and contains the PTK-like domain (grey box), and the rabbit IgG heavy chain (H), are indicated. No translation product of the PTK-like domain transcript produced in Ryk^{-/-} animals was detected between 16 and 400 kD (not shown). The right panel shows high-level expression of RYK β subunit in the MCF-7 cell line. The size of protein standards (in kD) is indicated. MAb, monoclonal antibody; GST.AF-6.PDZ, glutathione S-transferase fusion with the PDZ domain of mouse AF-6 (29).

FIG. 2 is a representation showing the phenotype of RYK-deficient mice. (a) Neonatal litternates illustrating the aerogastria (lower arrow), shortened mandible and snout (upper arrows) typical of RYK-deficient newborns. Note also the shortened forelmbs in the Ryk^{-/-} neonate (half arrows). (b) Growth retardation and cachexia in a Ryk^{-/-} mouse versus a Ryk^{+/-} littermate at postnatal day 7. (c) Female littermates at 6 months of age. The Ryk^{-/-} mouse showed growth retardation (34% of the bodyweight of the Ryk^{-/-} littermate shown), shortened snout, distorted skull shape, microphthalmia and an abnormal gait (splayed hindlimbs were dragged). (d) Alizarin red/alcian blue stained skull from a Ryk^{+/-} neonate. (e) Alizarin red/alcian blue stained skull from a Ryk^{-/-} newborn littermate illustrating the reduced head size, rounded cranial vault (cv), shortened mandible (m) and flattened face (f), involving shortened nasal bones and hypoplastic premaxilla and maxilla, characteristic of Ryk^{-/-} mice. Bones affected in RYK-deficient mice (e) are labelled on the skull from a heterozygous littermate (d) as follows; Nb, nasal bone; Fb, frontal bone; Pb, parietal bone; Ipb, interparietal bone, Sob, supraoccipital bone; Ma, mandible. (f) Full-body skeletal preparations of neonatal littermates (tail tips have been removed for genotyping). This Ryk^{-/-} animal (different from that shown in e) was severely reduced in size. In addition to the pronounced change in shape of the cranial vault, micrognathia and flattened face, Ryk^{-/-} neonates exhibited disproportionally shortened limbs (see Table 2) plus splayed hindliinbs (arrows). (g) Closed secondary palate in a Ryk^{+/-} neonate. (h) A complete cleft of the secondary palate (arrows) in a Ryk^{-/-} littermate which occurred with strong genetic penetrance (see text). (i) Illustration of developing mouse palate and tongue at 13.5-14.0 dpc in a schematic coronal section. (j) Coronal section of a Ryk^{+/-} embryo at 13.5 dpc showing strong β-galactosidase activity in the subepidermal mesenchynie of the tongue (arrow; scale bar, 250 μm). (k) Punctate pattern of β-galactosidase reporter activity in mesenchyme of a growing palatal shelf from a Ryk^{+/-} embryo at 13.5 dpc (arrowhead; scale bar, 100 μm), 0.5-1 day prior to palatal shelf fusion. (1, m, n, o, p) β-galactosidase histochemistry performed on frozen 30-40 μm coronal sections through the faces of embryos of the indicated genotypes at 13.5 (l-o) and 14.0 dpc (p). Arrows in o indicate staining associated with the tip of a vertical palatal shelf and the floor of the developing oral cavity. t, tongue; ps, palatal shelf; Ma, mandible. Scale bars (i-p), 50 μm. (q) Coronal sections through the midpalate region of wild-type and Ry^{-/-} embryos at the indicated developmental stages (in days post coitum). sp, secondary palate. Scale bars (l-w), 50 μm.

FIG. 3 is a representation showing interaction of RYK with Eph receptors. (a) Coprecipitation of EphB2, EphB3 and EphA7 with RYK from 293T cells transfected with the indicated expression plasmids. Only the full-length, unprocessed form of the RYK receptor is visualized in these experiments due to the location of the double Myc epitope tag at the RYK N-terminus. Levels of coprecipitated EphB2 were below the limit of detection with anti-EpbB2 antiserum, but the results of a reciprocal experiment (IP: α-EphB2, IB: α-Myc) indicated that small quantities of RYK were in fact coprecipitated with EphB2. α-pY, anti-phosphotyrosyl; IP, immunoprecipitation; IB, immunoblot; Myc2.RYK, double Myc epitope-tagged mouse RYK cDNA. (b) Tyrosyl phosphorylation of RYK is dependent on the PTK activity of EphB3. K323R and K665R, lysine to arginine substitution in PTK subdomain II of mouse RYK and human EphB3, respectively. (c) RTKs from other subfamilies do not coprecipitate with or induce tyrosyl phosphorylation of RYK. Tyrosyl-phosphorylated human EGFR, mouse VEGFR2 and mouse TIE2 were detectable in lysates but not anti-RYK immunoprecipitates from cotransfected 293T cells. (d) Coprecipitation of Eph receptors B2 and B3 with RYK from 12.5-13.5 dpc embryonic head lysates and wild-type adult brain lysates.

FIG. 4 is a representation showing interaction of RYK with the Ras target and Eph substrate AF-6. (a) RYK is coprecipitated with full-length AF-6. This interaction is dependent upon the RYK C-terminal valine residue and the PDZ domain of AF-6, but independent of any residual kinase activity which RYK may possess. (b) Co-expression of RYK, AF-6 and EphB3 or EphB2 in 293T cells. Anti-Myc immunoprecipitates contain the three proteins phosphorylated on tyrosyl. The presence of phosphotyrosyl in AF-6, RYK and EphB3 is critically dependent on the PTK activity of EphB3. (c) RYK and AF-6 are associated in vivo. Anti-AF-6 immunoprecipitates from 14.5 dpc embryonic head lysates contain RYK, while anti-RYK immunoprecipitates from wild-type adult brain lysates contain AF-6.

### DETAILED DESCRIPTION OF THE PREFERRED EMBODIMENTS

The present invention is predicated in part on the demonstration that the RYK receptor is required for normal development and morphogenesis of craniofacial structures including the secondary palate as well as being associated with other physiological conditions including neural conditions (e.g. aberrations in axon guidance or where axons fail to cross the midline such as in corpos callosum defects) and conditions affecting angiogenesis (e.g. blood vessels formation) and muscle development (e.g. muscle insertion) and maintenance. RYK may also be involved in the condition BPES as well as various tumours. BPES is a specific craniofacial syndrome which maps to human chromosome 3 which is close to the RYK gene. The identification of RYK's involvement in these conditions provides a means for the identification of particular abnormalities. The identification of RYK also provides a means for development of therapeutic agents including nucleic acid molecule-based therapeutics to ameliorate conditions exacerbated by the absence of a functional RYK receptor.

Accordingly, one aspect of the present invention contemplates a method for detecting a likelihood for progression of a developmental abnormality in an animal or for diagnosing the genetic or biochemical basis behind a particular developmental abnormality in said animal, said method comprising screening for the presence of a functional RYK molecule or its homologue or a mediator of RYK signalling or a functional nucleic acid molecule encoding said RYK or its signalling mediator or a transcript thereof wherein the absence of a functional RYK or its homologue or its signalling mediator or a mutation in the nucleic acid molecule encoding RYK or its signalling mediator or the presence of an aberration in the RYK transcript or signalling mediator transcript is indicative of or the likelihood of progression to a developmental disorder in said animal.

Reference herein to an animal is used in its broadest sense to include mammals, birds, fish, insects and reptiles. Preferred animals are humans such as primates, humans, livestock animals (e.g. cows, sheep, pigs, horses, donkeys), laboratory test animals (e.g. rabbits, mice, rats, hamsters), companion animals (e.g. dogs, cats) and captive wild animals. Most preferably, the animal is a primate or human or a laboratory test animal such as a mouse which provides extrapolatable data to humans.

The term “developmental disorder” is used in its broadest sense and encompasses the development of a range of organs, bone structures and other anatomically defined regions in unborn or early born animals. Most preferably, the development disorder involves aberrations in morphogenesis of craniofacial structures including the secondary palate as well as neural conditions (e.g. aberrations in axon guidance or where axons fail to cross the midline such as in corpos callosum defects) and conditions affecting angiogenesis (e.g. blood vessels formation) and muscle development (e.g. muscle insertion) and maintenance.

Accordingly, in a preferred embodiment, there is provided a method for detecting a likelihood for the progression of abnormal craniofacial structures in an animal or for diagnosing the genetic or biochemical basis behind a particular craniofacial abnormality or for detecting neurological conditions, conditions affecting angiogenesis and/or muscle development or maintenance in said animal, said method comprising screening for the presence of a functional RYK molecule or its homologue or a mediator of RYK signalling or a functional nucleic acid molecule encoding said RYK or its signalling mediator or a transcript thereof wherein the absence of a functional RYK or its homologue or its signalling mediator or a mutation in the nucleic acid molecule encoding RYK or its signalling mediator or the presence of an aberration in the RYK transcript or signalling mediator transcript is indicative of or the likelihood of progression to an abnormality in said animal.

A RYK signalling mediator is any component or member which is involved in RYK-mediated signalling or RYK-mediated function. In a particularly preferred embodiment, RYK signalling is mediated by Eph receptors and/or the cell function-associated AF-6 (Afadin). However, the present invention extends to RYK interaction with any other receptors including subfamilies of Eph receptors (e.g. EphB3, EphB6, EphA7 and EphB2 amongst others), and subfamilies of RYK as well as receptor tyrosine kinase families.

Detection of aberrations in RYK may be accomplished at the nucleic acid or protein levels.

At the nucleic acid level, nucleotide sequencing, differentiate probe hybridization and/or primer-mediated amplification iizter alia may be used to detect polymorphisms or other mutations in the RYK gene or in the gene encoding a RYK signalling mediator. The mutation screening approach SSCP is also effective. Other assays useful in detecting aberrations in gene or protein sequence include RNAse protection assays and protein:protein interaction assays. Microarrays (34) are particularly usefill in screening for particular mutations at the nucleotide or amino acid level. The presence of polymorphisms and other mutations provides an indication that the gene may be dysfunctional. A dysfunctional gene may either not be transcribed or may give rise to a transcript which cannot be translated into a functional protein. An aberrant RYK protein or aberrant RYK signalling mediator may also be detected at the protein level by, for example, screening for changes in immunological profile, physiochemical characteristics, electrophoretic characteristics or chromatographic characteristics.

The present invention extends, therefore, to nucleic acid molecules in the form of oligonucleotide probes or primers useful for detecting genomic sequences encoding an animal RYK molecule and in particular human RYK or a RYK signalling mediator. More particularly, the oligonucleotide probes are specific to particular regions of the genomic sequence such as those sequences encoding the extracellular domain, transmembrane domain or intracellular domain (including kinase catalytic domain) of RYK. Even more particularly, the oligonucleotide probes are useful in screening a genomic sequence for abnormalities in relation to the RYK coding sequence which result in an abnormal or mutant RYK which might in turn result in or facilitate RYK related abnormalities (e.g. in the morphogenesis of craniofacial structures).

Another aspect of the present invention contemplates an assay for identifying or otherwise diagnosing abnormalities in RYK or for identifying or otherwise screening for a normal RYK molecule in an animal such as a human. In accordance with this aspect of the invention, a source of genetic material is' isolated from an animal to be tested and subjected to any of a variety of assays such as Southern blot analysis, Northern blot analysis, Western blot analysis, radioimmunoassay (RIA) and other immunological techniques or variations or combinations of such analyses.

In one embodiment, there is provided a method for detecting an abnormal genomic coding sequence for a RYK or a protein having RYK-like properties in an animal subject, said method comprising contacting a genetic sample from said subject with one or more oligonucleotide primers specific for a part of the naturally occurring genomic sequence for said protein or for an abnormal coding sequence for said protein for a time and under conditions sufficient for said oligonucleotides to hybridize to said genomic sequence and then screening for said hybridization.

In a more particular embodiment, a subject is screened for a normal or abnormal RYK gene by isolating a genetic sample including genomic DNA or corresponding mRNA from said subject, subjecting said genetic sample to restriction endonuclease digestion to produce digested or partially digested DNA, subjecting said digested DNA to electrophoresis to separate the digested DNA based on length of fragments in the DNA digestion and screening the separated DNA digest to Southern blot analysis to screen for the presence or absence of particular regions of the RYK gene. For example, an oligonucleotide probe can be generated capable of screening for a nucleotide sequence corresponding to a “normal” extracellular region of RYK such as one or both of the leucine rich regions. In an abnormal RYK, the restriction pattern of this region may alter or contain deleted or duplicated sequences. Such an assay will screen for these modifications. Oligonucleotide probes may be designed to any other regions of RYK including transmembrane regions and intracellular regions.

An “abnormal RYK” is defined inter alia at the genetic level as an alteration in the nucleotide sequence encoding normal RYK such as to result in a RYK molecule with an altered amino acid sequence such as an insertion, deletion and/or substitution. The altered RYK may also have a different glycosylation pattern relative to the naturally occurring (i.e. normal) RYK molecule. Such a change in glycosylation patterns can result from a change in a single amino acid residue. An “abnormal RYK” can be defined inter alia at the functional level as a molecule having altered ligand binding characteristics. A “ligand” in this context includes any receptor, any protein partner or non-protein partner or any molecule involved in intermolecular binding. Frequently, this can result in abnormal development of craniofacial structures including the secondary palate. Mutations may also affect non-coding regions such as the 5′ or 3′ non-coding regions including the promoter and regulatory sequences. Mutations may also affect conformational changes of the RYK molecule and this may affect in turn the ability for RYK to interact with other molecules.

Aberrant RYK proteins or other proteins associated with RYK signalling may also be detected immunologically. The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art (31, 32).

This aspect of the present invention is predicated in part, therefore, on the discovery that aberrations in RYK signalling are associated with disease conditions. Thus, the present invention contemplates a method for diagnosis in a patient of a disease condition, or of the probable affliction therewith, comprising detecting an aberrant gene or aberrant expression of a gene encoding a RYK in a biological sample obtained from said patient.

In one embodiment, the method comprises detecting a change in the level and/or functional activity of a target molecule selected from the group consisting of an expression product of the RYK gene and an expression product of another gene relating to the same regulatory or biosynthetic pathway as the RYK gene, wherein the change is relative to a normal reference level and/or functional activity. For example, the presence of, or the probable affliction with, a cancer or tumour is diagnosed when the RYK gene product is altered relative to a normal control. In a preferred embodiment of this type, the method comprises detecting a level and/or functional activity of an expression product of the RYK gene.

Thus, it will be desirable to qualitatively or quantitatively determine RYK protein levels and/or RYK transcription levels. Alternatively or additionally, it may be desirable to search for an aberrant RYK gene and/or regulatory regions. Alternatively or additionally, it may be desirable to qualitatively or quantitatively determine the level of an expression product (e.g. transcript, protein) of a gene relating to the same regulatory or biosynthetic pathway as the RYK gene, which can modulate or otherwise influence RYK protein levels and/or RYK transcription levels. Likewise, it may also be desirable to search for an aberrant gene relating to the same regulatory or biosynthetic pathway as a RYK gene.

The biological sample can include any suitable tissue or fluid. Suitably, the biological sample is a tissue biopsy, preferably selected from kidney, brain, and testis.

Nucleic acid used in polynucleotide-based assays can be isolated from cells contained in the biological sample, according to standard methodologies (Sambrook, et al., “Molecular Cloning. A Laboratory Manual”, Cold Spring Harbor Press, 1989; Ausubel et al., “Current Protocols in Molecular Biology”, John Wiley & Sons Inc, 1994-1998). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. In one embodiment, the nucleic acid is amplified by a nucleic acid amplification technique. Suitable nucleic acid amplification techniques are well known to the skilled addressee, and include the polymerase chain reaction (PCR); strand displacement amplification (SDA) as, for example, described in U.S. Pat. No. 5,422,252; rolling circle replication (RCR) as described, for example, in International application WO 92/01813 and International Application WO 97/19193; nucleic acid sequence-based amplification (NASBA) and Qβ replicase amplification.

Depending on the format, the specific nucleic acid of interest is identified in the sample directly using amplification or with a second, known nucleic acid following amplification. Next, the identified product is detected. In certain applications, the detection may be performed by visual means (e.g. ethidium bromide staining of a gel). Alternatively, the detection may involve indirect identification of the product via chemiluminescence, radioactive scintigraphy of radiolabel or fluorescent label or even via a system using electrical or thermal impulse signals.

Following detection, one may compare the results seen in a given patient with a control reaction or a statistically significant reference group of normal patients. In this way, it is possible to correlate the amount of a RYK detected with the progression or severity of the disease.

These defects or other aberrations in the RYK include deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of RYK produced, both by altering the transcription of the gene or in destabilizing or otherwise altering the processing of either the transcript (mRNA) or protein.

A variety of different assays are contemplated in this regard, including but not limited to, fluorescent in situ hybridiztion (FISH), direct DNA sequencing, pulse field gel electrophoresis (PFGE) analysis, Southern or Northern blotting, single-stranded conformation analysis (SSCA), RNase protection assay, allele-specific oligonucleotide (ASO), dot blot analysis, denaturing gradient gel electrophoresis, RFLP and PCR-SSCP.

Primers may be provided in double-stranded or single-stranded form, although the single-stranded form is preferred. Probes, while perhaps capable of priming, are designed to bind to a target DNA or RNA and need not be used in an amplification process. In preferred embodiments, the probes or primers are labelled with radioactive species ³²P, ¹⁴C, ³⁵S, ³H, or other label), with a fluorophore (rhodamine, fluorescein) or a chemillumiscent label (luciferase).

A number of template dependent processes are available to amplify the marker sequences present in a given template sample. An exemplary nucleic acid amplification technique is the polymerase chain reaction (referred to as PCR) which is described in detail in U.S. Pat. Nos. 4,683,195, 4,683,202 and 4,800,159.

Briefly, in PCR, two primer sequences are prepared that are complementary to regions on opposite complementary strands of the marker sequence. An excess of deoxynucleoside triphosphates are added to a reaction mixture along with a DNA polymerase, e.g. Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al, 1989 (33). Alternative methods for reverse transcription utilise thermostable, RNA-dependent DNA polymerases. These methods are described in WO 90/07641. Polymerase chain reaction methodologies are well known in the art.

Another method for amplification is the ligase chain reaction (“LCR”), disclosed in EPO No. 0 320 308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qβ Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′α-thio-triphosphates in one strand of a restriction site may also be useful in the amplification of nucleic acids in the present invention.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2,202,328, and in PCT Application No. PCT/US89/01025, may be used in accordance with the present invention. In the former application, “modified” primers are used in a PCR-like, template- and enzyme-dependent synthesis. The primers may be modified by labelling with a capture moiety (e.g. biotin) and/or a detector moiety (e.g. enzyme). In the latter application, an excess of labelled probes are added to a sample. In the presence of the target sequence, the probe binds and is cleaved catalytically. After cleavage, the target sequence is released intact to be bound by excess probe. Cleavage of the labelled probe signals the presence of the target sequence.

Other nucleic acid amplification procedures include transcription-based amplification systems (TAS), including nucleic acid sequence based amplification (NASBA) and 3SR (PCT Application WO 88/10315). In NASBA, the nucleic acids can be prepared for amplification by standard phenol/chloroform extraction, heat denaturation of a clinical sample, treatment with lysis buffer and mini-spin columns for isolation of DNA and RNA or guanidinium chloride extraction of RNA. These amplification techniques involve annealing a primer which has target specific sequences. Following polymerisation, DNA/RNA hybrids are digested with RNase H while double stranded DNA molecules are heat denatured again. In either case the single stranded DNA is made fully double stranded by addition of second target specific primer, followed by polymerisation. The double-stranded DNA molecules are then multiply transcribed by an RNA polymerase such as T7 or SP6. In an isothermal cyclic reaction, the RNAs are reverse transcribed into single stranded DNA, which is then converted to double stranded DNA, and then transcribed once again with an RNA polymerase such as T7 or SP6. The resulting products, whether truncated or complete, indicate target specific sequences.

European Patent No. 0 329 822 discloses a nucleic acid amplification process involving cyclically synthesising single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

In International Application WO 89/06700, there is disclosed a nucleic acid sequence amplification scheme based on the hybridisation of a promoter/primer sequence to a target single-stranded DNA (“ssDNA”) followed by transcription of many RNA copies of the sequence. This scheme is not cyclic, i.e. new templates are not produced from the resultant RNA transcripts. Other amplification methods include “RACE” and “one-sided PCR”.

Methods based on ligation of two (or more) oligonucleotides in the presence of nucleic acid having the sequence of the resulting “di-oligonucleotide”, thereby amplifying the dioligonucleotide, may also be used in the amplification step of the present invention.

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labelled) under conditions that promote denaturation and rehybridization. Because the probe is designed to base pair with the target, the probe will binding a portion of the target sequence under renaturing conditions. Unbound probe is then removed, and detection is accomplished as described above.

Products may be visualized in order to confirm amplification of the marker sequences. One typical visualisation method involves staining of a gel with ethidium bromide and visualisation under UV light. Alternatively, if the amplification products are integrally labelled with radio- or fluorometrically-labelled nucleotides, the amplification products can then be exposed to x-ray film or visualised under the appropriate stimulating spectra, following separation.

In one embodiment, visualization is indirectly achieved. Following separation of amplification products, a labelled nucleic acid probe is brought into contact with the amplified marker sequence. The probe preferably is conjugated to a chromophore but may be radiolabelled. In another embodiment, the probe is conjugated to a binding partner, such as an antibody or biotin, and the other member of the binding pair carries a detectable moiety or reporter molecule.

In one embodiment, detection is by a labelled probe. The techniques involved are well known to those of skill in the art and can be found in many standard texts on molecular protocols. See Sambrook et al., 1989. For example, chromophore or radiolabel probes or primers identify the target during or following amplification.

One example of the foregoing is described in U.S. Pat. No. 5,279,721, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing. The present invention provides methods by which any or all of these types of analyses may be used. Using, for example, the sequences set forth in herein, oligonucleotide primers may be designed to permit the amplification of sequences throughout RYK that may then be analysed by direct sequencing.

All the essential materials and reagents required for detecting and sequencing RYK or a RYK gene and variants thereof may be assembled together in a kit. The kits may also optionally include appropriate reagents for detection of labels, positive and negative controls, washing solutions, dilution buffers and the like. For example, a nucleic acid-based detection kit may include (i) a polynucleotide according to the invention (which may be used as a positive control), (ii) an oligonucleotide primer according to the invention. Also included may be enzymes suitable for amplifying nucleic acids including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe.

Also contemplated by the present invention are chip-based DNA technologies. Briefly, these techniques involve quantitative methods for analysing large numbers of genes rapidly and accurately. By tagging genes with oligonucleotides or using fixed probe arrays, one can employ chip technology to segregate target molecules as high density arrays and screen these molecules on the basis of hybridization.

Another aspect of the present invention contemplates a method for detecting a non-abnormal RYK in a biological sample from a subject said method comprising contacting said biological sample with an antibody specific for functional RYK or its derivatives or homologues for a time and under conditions sufficient for an antibody-RYK complex to form, and then detecting said complex.

The presence of RYK may be accomplished in a number of ways such as by Western blotting and ELISA procedures. A wide range of immunoassay techniques are available as can be seen by reference to U.S. Pat. Nos. 4,016,043, 4,424,279 and 4,018,653. These, of course, includes both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention, the sample is one which might contain RYK including cell extract, tissue biopsy or possibly serum, saliva, mucosal secretions, lymph, tissue fluid, respiratory fluid and CSF. The sample is, therefore, generally a biological sample comprising biological fluid but also extends to fermentation fluid and supernatant fluid such as from a cell culture.

In a typical forward sandwich assay, a first antibody having specificity for the RYK or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. room temperature to 40° C. including 25° C., 30° C. and 37° C.) to allow binding of any subunit present in the antibody. Following the incubation period, the antibody subunit solid phase is washed and dried and incubated with a second antibody specific for a portion of the RYK. The second antibody is linked to a reporter molecule which is used to indicate the binding of the second antibody to the RYK.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody.

Alternatively, a second labelled antibody, specific to the first antibody is exposed to the target-first antibody complex to form a target-first antibody-second antibody tertiary complex. The complex is detected by the signal emitted by the reporter molecule.

By “reporter molecule”, as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody RYK complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of RYK which was present in the sample. “Reporter molecule” also extends to use of cell agglutination or inhibition of agglutination such as red blood cells on latex beads, and the like.

Alternately, fluorescent compounds, such as fluorecein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. As in the EIA, the fluorescent labelled antibody is allowed to bind to the first antibody-RYK complex. After washing off the unbound reagent, the remaining tertiary complex is then exposed to the light of the appropriate wavelength, the fluorescence observed indicates the presence of the RYK of interest. Immunofluorescene and EIA techniques are both very well established in the art and are particularly preferred for the present method. However, other reporter molecules, such as radioisotope, chemiluminescent or bioluminescent molecules, may also be employed.

Any number of variations may be pursued including using antibodies to particular RYK mutants and using differentiated antibody display library techniques. Furthermore, the method may also be conducted by the immunological screening of aberrant mediators of RYK signalling. RYK may also be assayed on the basis of its ability or inability to interact with particular ligands such as receptors. For example, interation with an Eph receptor may be used to determine whether or not RYK is aberrant. Although not intending to limit the present invention to any one theory or mode of action, another ligand which RYK may interact with is Wnt. It is known that the RYK receptor comprises a Wnt-inhibitory factor 1 (W1F-1) molecule (33). WIF-1 molecules bind to Wnt and hence RYK may also interact with Wnt proteins. The absence of interaction with Wnt may be an indicator of an aberrant RYK.

The term “aberrant” is used in its broadest sense to include single or multiple nucleotide or amino acid substitutions, additions and/or deletions to the RYK gene or the RYK molecule as well as to non-coding regions such as the promotor, enhancer or other regulatory regions. Generally, such mutations would result in the physiological effects of craniofcial structure abnormalities as well as neural conditions (e.g. aberrations in axon guidance or where axons fail to cross the midline such as in corpos callosum defects) and conditions affecting angiogenesis (e.g. blood vessel formation) and muscle development (e.g. muscle insertion) and maintenance.

The subject is preferably human and may be an adult, adolescent, child, infant or a foetus.

The assay may be particularly useful in screening members of a family with a pre-disposition to craniofacial abnormalities based on a defective or modified RYK molecule.

The nucleotide and corresponding amino acid sequences of human and mouse RYK are shown in International Patent Application No. PCT/AU93/00210 (WO 93/23429) which is incorporated herein by reference. RYK is also defined in references 4-6 and 26.

The identification of RYK as an important component in normal craniofacial structure development as well as neural conditions (e.g. aberrations in axon guidance or where axons fail to cross the midline such as in corpos callosum defects) and conditions affecting angiogenesis (e.g. blood vessel formation) and muscle development (e.g. muscle insertion) and maintenance. RYK may also have a role in tumourigenesis. The identification of RYK also provides a means for gene therapy or even biochemical intervention. Abnormalities detected, for example, in utero may provide an opportunity to introduce a RYK gene or to introduce a gene encoding a RYK signalling mediator or to introduce a protein having RYK activity to facilitate normal signalling and thereby normal craniofacial structure development as well as neural conditions (e.g. aberrations in axon guidance or where axons fail to cross the midline such as in corpos callosum defects) and conditions affecting angiogenesis (e.g. blood vessel formation) and muscle development (e.g. muscle insertion) and maintenance. In one embodiment, the present invention provides a genetic construct encoding RYK or a mediator of RYK signalling for use in direct administration to appropriate tissue.

Alternatively, RYK protein may be administered. Yet in another alternative, animal models such as mice having a Ryk^{-/-} or Ryk^{+/-} phenotype are used to screen chemical and natural product libraries for molecules which block, reverse or otherwise ameliorate the effects of the mutated Ryk phenotype. All such molecules identified by this approach are encompassed by the present invention.

Accordingly, another aspect of the present invention is directed to a composition comprising a nucleotide sequence encoding RYK or a homologue thereof or a mediator of RYK signalling or functional derivatives thereof, said nucleotide sequence operably linked to a promoter or functional derivative, homologue or hybrid form wherein said genetic construct when introduced into an animal cell is capable of directing the production of RYK or a mediator of RYK signalling said composition comprising one or more pharmaceutically acceptable carriers and/or diluents.

Such a composition may also be referred to as a pharmaceutical composition.

The compositions are preferably in a form suitable for administration by injection, infusion, implant, needleless injection, drip, oral intake and/or electrotransfer. The composition may also be in a form suitable for intake via inhalation or nasal spray.

Composition forms suitable for injectable use include sterile aqueous solutions. These must be stable under the conditions of manufacture and storage and must be preserved against the contaminating action of micro-organisms such as bacteria and fungi. The carrier can be a solvent or diluent containing, for example, water, ethanol, polyol (for example, glycerol, propylene glycol and liquid polyethylene glycol and the like), suitable mixtures thereof and vegetable oils. The preventions of the action of micro-organisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Pharmaceutically acceptable carriers and/or diluents include any and all solvents, dispersion media, coatings, antibacterial and antifingal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

A single dose may be administered or multiple doses over time such as over hours, days, weeks or months. Preferably, the constructs are administered to non-pancreatic cells. Alternatively, cells carrying the genetic constructs may be maintained in in vitro culture prior to transplanting or otherwise introducing the cells into a recipient.

Amounts of construct required will vary depending on the subject and the level of RYK needed. However, dosage generally from about 1 μg to about 100 mg of genetic construct per injection or from about 10 μg to about 10 mg of genetic construct per injection may be employed. Alternatively, the amount administered may be expressed in amounts per kilogram of body weight. Accordingly, the effective amount maybe from about 0.01 μg to about 10 mg/kg body weight or more preferably from about 0.1 μg to about 5 mg/kg body weight.

Less construct may be administered where a strong promoter, for example, or other expression enhancers, permit high level production of insulin. Accordingly, the amount of construct may be that which provide from about 0.01 μg to about 10 mg of RYK/kg body weight of recipient.

Yet another aspect of the present invention contemplates genetically modified animals which are deficient in or on both Ryk alleles. Preferably, the animal is a laboratory test animal such as a mouse, rat, rabbit or hamster. Such animals are useful models for craniofacial abnormalities.

It is a further within the scope of the present invention to encompass genotypes which mask the effects of a RYK deficiency. Such genotypes may result from expression of a particular nucleotide sequence or loss of expression of a particular nucleotide sequence which in turn may be a useful means to treat RYK deficiency. Such a genotype may be identified in any number of ways including crossing a mutagenesized mouse or other animal with a non-mutageneized mouse to develop a G₁ founder mouse. This is then outcrossed with an index strain such as a strain carrying a mutation in RYK and which exhibits an identifiable phenotype. The resulting G₁F₁ kindreds are then screened for the presence or absence of a modification to the index phenotype. A parent mouse may then be used to clone the mutation affecting the RYK deficiency. In a modified protocol, the female parent founder mouse giving rise to a modified outlying phenotype is crossed to a non-mutagenesized mouse to produce a copy generation (G₂). This is then crossed with the index strain and kindreds with a modified phenotype identified. These are then used to clone the modifying genetic sequences.

Still yet another aspect of the present invention contemplates the use of Ryk or RYK or a mediator of RYK signalling in the manufacture of a medicament or diagnostic agent for the treatment or diagnosis of development abnormalities in an animal including neural conditions (e.g. aberrations in axon guidance or where axons fail to cross the midline such as in corpos callosum defects) and conditions affecting angiogenesis (e.g. blood vessel formation) and muscle development (e.g. muscle insertion) and maintenance.

Preferably, the development abnormalities is a craniofacial structure abnormality.

The present invention is further described by the following non-limiting Examples.

### EXAMPLE 1

### Gene Targeting of Ryk

The genomic structure of mouse Ryk has been previously reported (5). The inventors prepared a promoterless targeting vector by flanking an IRES.βgeo.pA expression cassette in pbluescript II KS(−) with long (7.2 kb KspI-NotI fragment) and short (1.2 kb KpnI fragment) arms of homology. The KspI site in the long arm was derived from a pBluescript II KS(+) polylinker and the NotI site was introduced into exon 2 by site-directed mutagenesis. The short arm was isolated as a 1.2 kb KpnI fragment (3′ KpnI site derived from a pBluescript II KS(+) polylinker), shuttled into pIC 20 and excised as a SacI fragment for cloning into the targeting vector. The inventors electroporated the W9.5 ES cell line with targeting vector linearized at the unique KspI site flanking the long arm of homology and screened genomic DNA extracted from colonies surviving G418 selection for targeting events by Southern blot analysis using a XbaI digest and 3′ flanking probe. The inventors further tested targeted clones subsequently used to generate chimeric mice by Southern blotting using a 5′ flanking probe and a βgeo-specific probe to confirm a crossover on the long arm of homology and genomic integration at a single site, respectively. The inventors used two independently targeted ES cell clones to generate chimeric mice that subsequently transmitted the mutation through the germline. The phenotype of Ryk^{-/-} mice derived from the two targeted ES cell lines was indistinguishable, and the results presented here represent analysis of the RKO.44 strain on a hybrid 129/Sv×C57BL/6J genetic background.

### EXAMPLE 2

### Genotyping of Mice

The inventors genotyped mice by two-allele, three-primer PCR using genomic DNA template prepared from tail clips or embryonic tissues. Touchdown PCR was used to generate products of 505 bp from the wild type Ryk allele and/or a product of 625 bp from the targeted Ryk allele. Primers used were specific for the βgeo cassette (sense, GTKO3, 5′-GCGTTGGCTACCCGTGATA-3′ [SEQ ID NO:1]), intron 6 common to the wild type and targeted Ryk alleles (antisense, GTR, 5′-CAAGTAACATGCTCCCCAAAAC-3′ [SEQ ID NO:2]) and intron 5 deleted from the targeted Ryk allele (sense, GTwt, 5′-CAGGGCAGTGCATTCCATCT-3′ [SEQ ID NO:3]).

### EXAMPLE 3

### RT-PCR

The inventors synthesized cDNA from 5 μg of DNase I-treated total RNA isolated from whole E18.5 embryos (RNeasy, Qiagen) using a First Strand Synthesis for RT-PCR Kit (Amersham) and used one-tenth of the total cDNA as template in touchdown multiplex PCR reactions containing primers to the mouse Ryk exodomain/transmembrane domain (sense, 5′-TTGTGGCTATGGGCATGC-3′ [SEQ ID NO:4]; antisense, 5′-GAAATATCACTGCACAGC-3′ [SEQ ID NO:5]; 336 bp product), kinase-like domain (sense, 5′-CATATGGCTATTCAGATTG-3′ [SEQ ID NO:6]; antisense, 5′-TGGACCAGCTGCTGGAACTAA-3′ [SEQ ID NO:7]; 451 bp product) and the βgeo transgene (sense, 5′-GACTGTGGCCGGCTGGGTGTG-3′ [SEQ ID NO:8]; antisense, 5′-GGGCGTCGCTTGGTCGGTCATT-3′ [SEQ ID NO:9]; 215 bp product).

### EXAMPLE 4

### Histology, Whole Mount Skeletal Staining and β-Gal Histochemistry

For histological analysis, the inventors used perfusion-fixed samples with cold 4% v/v paraformaldehyde in PBS (pH 7.3), processed them into serial paraffin-embedded sections and stained with haematoxylin and eosin. The inventors performed whole mount skeletal staining and histochemical analysis of β-galactosidase reporter activity in frozen sections (counterstained with nuclear fast red) as described previously (28).

### EXAMPLE 5

### Expression Constructs

To produce Myc2.RYK, the inventors cloned a PCR fragment (BssHII-KspI) encoding an ideal Kozak sequence, mouse IL-3 signal sequence and double Myc epitope tag into pcDNA3 (Invitrogen) upstream and in frame with the predicted mature N-terminus of mouse RYK. The inventors produced mutants of Myc2.RYK (K323R and V594A) using the QuikChange Site-Directed Mutagenesis Kit (Stratagene). FLAG epitope-tagged AF-6 and AF-6.PDZ, GST.AF-6.PDZ, and the human EphB2, EphB3 and EphA7 cDNAs have been previously described (13, 29). The inventors cloned the human EGFR cDNA into pcDNAI/Amp (Invitrogen) and mouse TIE2 and VEGFR2 cDNAs into the pCDM8 expression vector (Invitrogen).

### EXAMPLE 6

### Immunoprecipitation and Western blotting

The inventors transiently transfected 10 or 15 cm dishes of 293T cells cultured in DMEM, 10% w/v FCS, 2 mM glutamine and 50 μg/mL gentamycin with a total of 5-15 μg of plasmid DNA (overexpression of some cDNAs was toxic and the amount transfected required titration) using FuGene 6 (Boehringer Mannheim) and lysed cells on the plate 24-36 hrs later in 1 ml of 50 mM HEPES pH 7.4, 150 mM NaCl, 0.5-1% Triton X-100, 10% v/v glycerol (HNTG) supplemented with 2 mM activated Na₃VO₄, 2 mM NaF and Complete Mini Protease Inhibitor Cocktail (Boehringer Mannheim). Lysis and subsequent analysis of complexes in triple-transfected cells was performed as previously described (13). The inventors prepared embryonic and tissue lysates by Dounce homogenization. The inventors cleared lysates of insoluble debris by centrifugation, and performed immunoprecipitation from cleared lysates containing equal amounts of total protein as assessed by the BCA assay (Pierce). Immune complexes were captured by incubation with protein A-, G- or L-conjugated beads. The mouse anti-RYK monoclonal antibody (IgMₓ isotype⁵) was covalently coupled to CNBr-activated Sepharose 4B (Pharmacia) at 1 mg/ml packed beads for immunoprecipitation or cross-linked it in solution using a goat anti-mouse IgM (Jackson Immunoresearch). The inventors used anti-FLAG monoclonal antibody M2 (Sigma) for immunoprecipitation as an affinity gel and for Western blotting in a biotinylated form. Anti-AF-6 monoclonal antibody (clone 35) was obtained from Transduction Laboratories and anti-Myc monoclonal antibody (clone 9E10) from Zymed Laboratories. Immune complexes were collected by centrifugation and washed three times in HNTG, eluted in 20-50 μL of 2×SDS-PAGE sample buffer containing 10% v/v 2-mercaptoethanol, fractionated by SDS-PAGE and electroblotted to Immobilon P (Millipore) for Western analysis. Goat anti-human EphB2 antiserum (C-20) and rabbit anti-mouse EphA7 antisera (K-16, raised against an epitope at the amino terminus; C-19, raised against an epitope at the carboxyl terminus) were obtained from Santa Cruz Biotechnology; anti-EphB3 antiserum has been described previously (13) or was purchased from R&D systems. The inventors employed HRP-conjugated monoclonal antibody 4G10 (Upstate Biotechnology) to detect phosphotyrosyl. The following antibodies were used against irrelevant RTKs: anti-TIE2 monoclonal antibody (30), rabbit anti-VEGFR2 antiserum (R. B. Oelrichs, A. F. W. and S. A. S., unpublished) and sheep anti-human EGFR antiserum (Dr. Hongjian Zhu, LICR, Melbourne). The GST.AF-6.PDZ fusion protein from pET.GEX-9.1 was expressed in E. coli BL21(DE3); it was purified by glutathione-sepharose affinity chromatography and used for blot analysis at ˜5 jug/mL in PBS containing 5% w/v skim milk, 1% v/v Triton X-100 and detected bound GST.AF-6.PDZ with rabbit anti-GST polyclonal antibody (Upstate Biotechnology) followed by HRP-conjugated goat anti-rabbit IgG (Bio-Rad). All Western blots were developed using SuperSignal West Pico Chemiluminescent Substrate (Pierce).

### EXAMPLE 7

### Effects of Ryk Detection

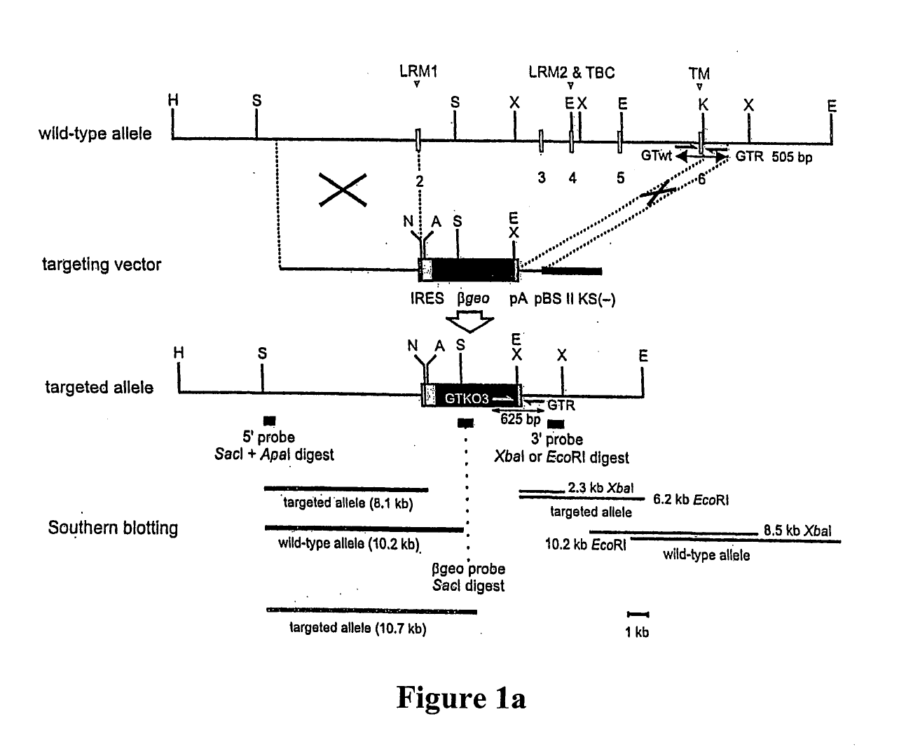

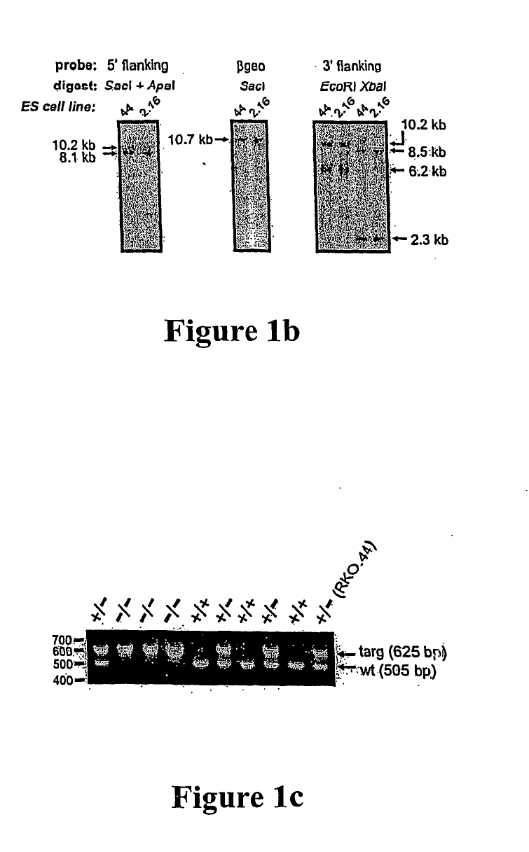

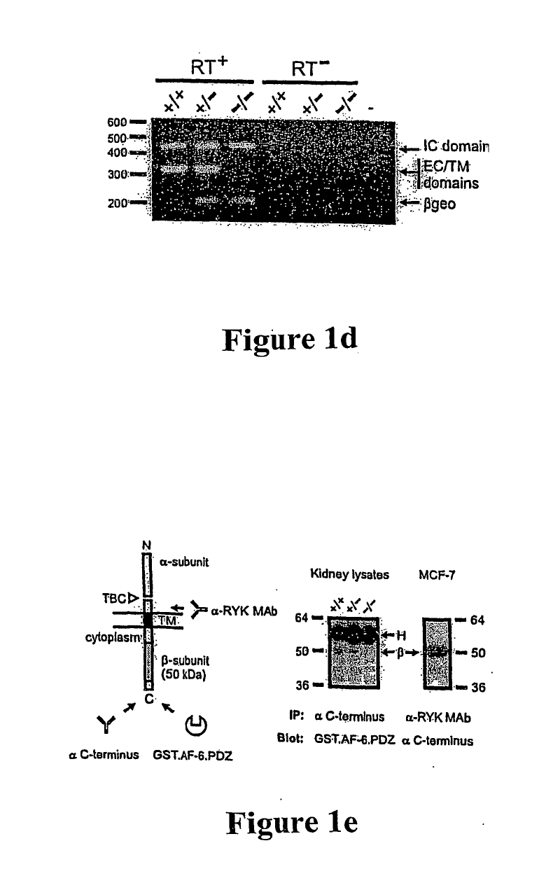

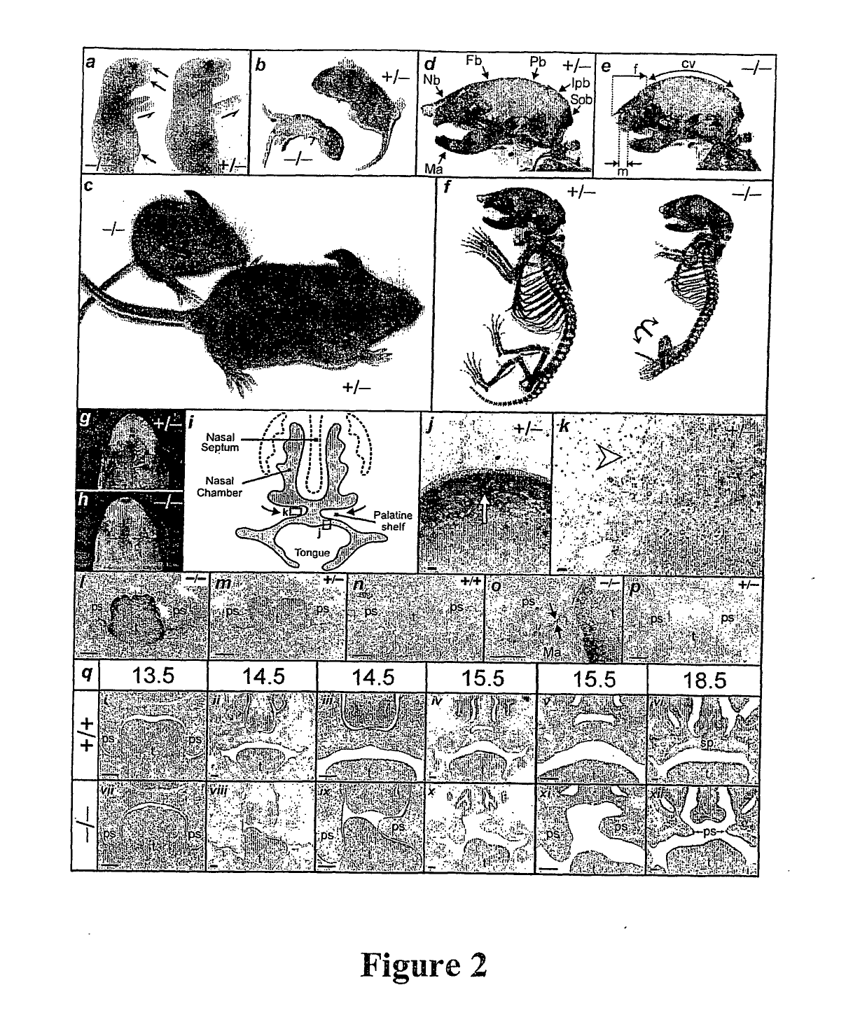

The inventors generated a 14.5 kb deletion in Ryk by gene targeting (FIG. 1a, b, c). RT-PCR and blotting analysis of anti-RYK immunoprecipitates (FIG. 1d, e) indicated that the targeted Ryk allele represents a null mutation. Genotyping of offspring from Ryk^{+/-} intercrosses late in gestation, immediately postpartum and at weaning (Table 1) showed that Ryk^{-/-} mice were represented in Mendelian proportions up to birth, but virtually none survived to weaning. Ryk^{-/-} neonates often failed to suckle and soon became dehydrated and cyanotic, exhibiting gasping respirations and air-distended stomachs (FIG. 2a).

Ryk^{-/-} mice typically died on the day of birth, while a few survivors became increasingly cachexic (FIG. 2b) and died before 8 days. Two Ryk^{-/-} mice survived to adulthood, but were severely growth retarded and exhibited microcephaly, bilateral microphthalmia (FIG. 2c) and an unusual gait involving dragging of the hindlimbs. Brains from Ryk^{-/-} mice were grossly normal.

While Ryk^{+/-} mice were indistinguishable from their wild-type littermates, all Ryk^{-/-} neonates showed a shortened snout (FIG. 2a) and a completely cleft secondary palate (FIG. 2g, h) which occurred with a genetic penetrance of >88% (31 of 35 Ryk^{-/-} versus 0 of 160 wild-type and heterozygous littermates). In addition, the Ryk^{-/-} mice displayed disproportionally shorter limbs, with the hindlimbs splayed laterally (FIG. 2a, f). The cranial vault in newborn Ryk^{-/-} mice was slightly smaller and more rounded than in wild-type or heterozygous mice, reflecting the sum of minor changes in size and shape of individual calvarial elements (FIG. 2d, e). The mandible was also significantly reduced in size (Table 2; FIG. 2d, e, f). The facial skeleton was more strongly affected (FIG. 2 d, e; Table 2), the shortened snout and flattened midface reflecting significantly shortened nasal bones and premaxillary/maxillary hypoplasia, respectively (Table 2). The reduction in size of these facial bones was not uniform in all axes because the face was of normal width. No obvious craniofacial patterning defects, manifested as homeosis or aplasia, were evident in Ryk^{-/-} neonates. The limbs of Ryk^{-/-} neonates (FIGS. 2a, f) were affected similarly to the facial skeleton; the long bones were reduced in length by 18-25% (Table 2) but were of normal or slightly greater than normal width. Combined with the strong expression of Ryk mRNA in 13 dpc embryonic rat limb buds (10), this finding reveals a vital function for RYK in limb buds which share many patterning and morphogenetic mechanisms with the developing facial primordia (3).

The inventors performed β-galactosidase histochemistry on sections of the developing facial complex to reveal Ryk promoter activity. Intense staining of the tongue at 12.5-13.5 days post coitum (dpc), particularly the subepidermal mesenchyme (FIG. 2i, j, l, m, o), and weaker staining of palatal shelf mesenchyme (FIG. 2i, k). Also strongly stained were the tips of the vertical palatal shelves and the floor of the developing oral cavity at 13.5 dpc (FIG. 21, m, o). Ryk promoter activity was strongly downregulated by 14.0 dpc (FIG. 2p).

To determine the cause of cleft palate in RYK-deficient neonates, the inventors examined coronal sections through the midpalate region of wild-type and Ryk^{-/-} embryos at 13.5, 14.5, 15.5 and 18.5 dpc (FIG. 2q). Downward growth of the vertical palatal shelves at 13.5 dpc in wild-type and Ryk^{-/-} embryos was indistinguishable (FIG. 2q i, vii). However, by 14.5 dpc the tongue consistently obstructed physical elevation of one of the two palatal shelves in Ryk^{-/-} embryos (FIG. 2q viii, ix), preventing their contact at the midline and subsequent closure of the secondary palate (FIG. 2q x-xii). Together with the ability of palatal shelves from 12.5 dpc Ryk^{-/-} embryos to fuse in vitro (not shown), these findings strongly suggest that the cleft palate observed in Ryk^{-/-} neonates is a secondary consequence of defective extra-palatal craniofacial morphogenesis. Transient trapping of an elevating palatal shelf in Ryk^{-/-} embryos apparently results from abnormally high positioning of the tongue, which appears slightly enlarged, at 13.5-14.5 dpc. The strong Ryk promoter activity detected in the tongue and floor of the developing oral cavity are consistent with a requisite role for RYK in correct positioning of the tongue during secondary palate formation.

Mice simultaneously deficient in EphB2 and EphB3 exhibit commissural axon pathfinding defects and a cleft palate with strikingly similar morphology and penetrance to that observed in Ryk^{-/-} neonates (7). In addition, a Drosophila orthologue of RYK, Derailed, functions embryonically as a commissural axon guidance receptor (8, 9). These findings led the inventors to explore the possibility of functional interaction between RYK and Eph receptors. The expression of Ryk in developing palatal shelves and tongue (FIGS. 2i-o) overlaps temporospatially with EphB2, EphB3 and EphA7 (7, 11), consistent with the possibility of physical interaction between RYK and these Eph receptors in vivo.

### EXAMPLE 8

### Interaction of RYK with Eph Receptors

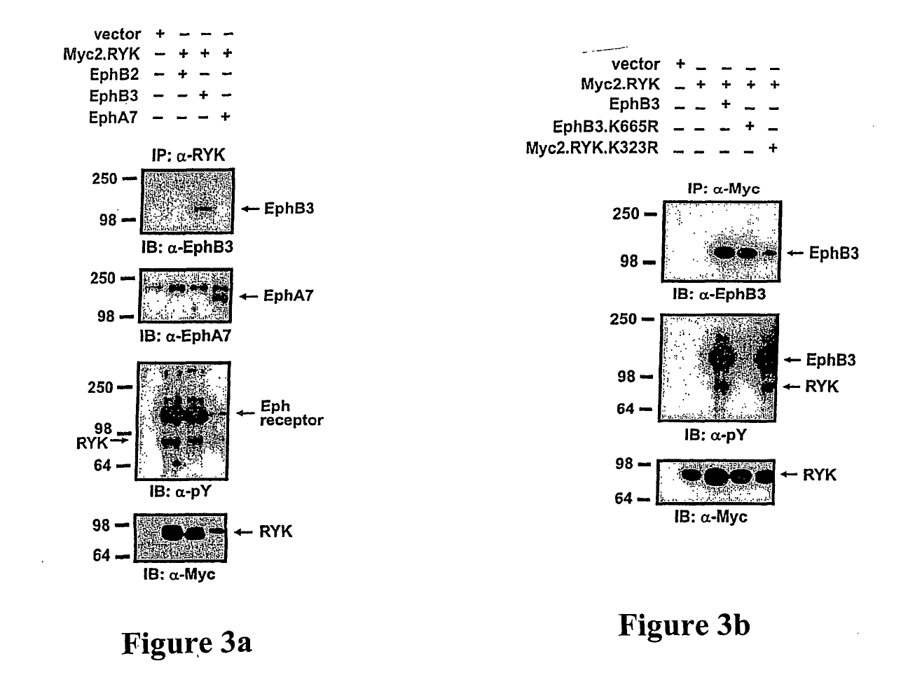

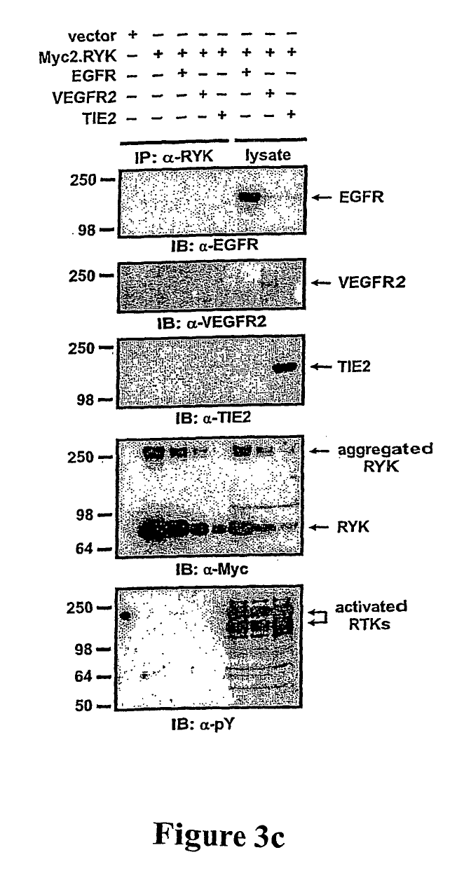

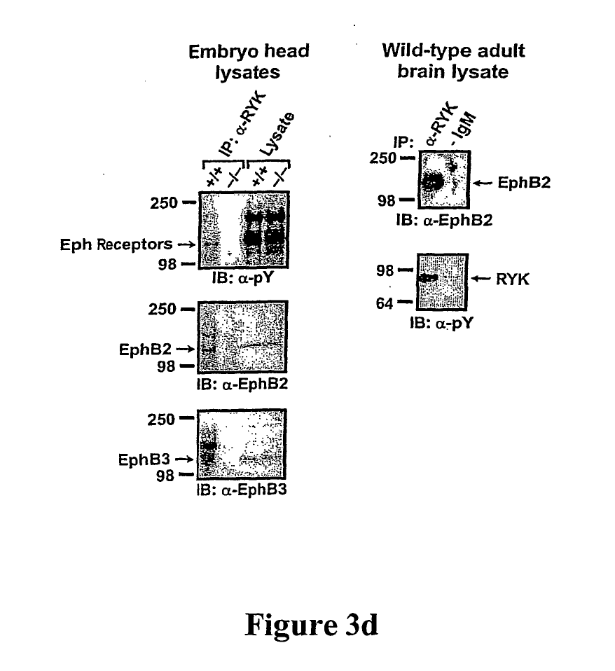

To investigate the potential for interaction of RYK with Eph receptors, in vitro experiments were performed. The inventors observed coprecipitation of EphB2, EphB3 and EphA7 with epitope-tagged RYK from cotransfected 293T cells (FIG. 3a). RYK was tyrosyl-phosphorylated when coexpressed with EphB2 or EphB3, but not EphA7 (FIG. 3a). When coexpressed with a kinase-defective mutant of EphB3 (K665R, FIG. 3b), RYK was no longer tyrosyl-phosphorylated, signifying that phosphorylation of RYK is critically dependent upon the kinase activity of EphB3. This represents the first observation of tyrosyl-phosphorylated RYK and implicates this catalytically inactive RTK in signalling mediated by particular Eph receptors. In contrast, other RTKs (EGFR, VEGFR2 and TIE2; EphA3) could not be coprecipitated with or induce tyrosyl-phosphorylation of RYK when coexpressed in vitro (FIG. 3c). Consistent with these results, in anti-RYK immunoprecipitates from wild-type, but not Ryk^{-/-}, 13.5 dpc head lysates, Eph receptors B2 and B3 were observed to coprecipitate with RYK (FIG. 3d).

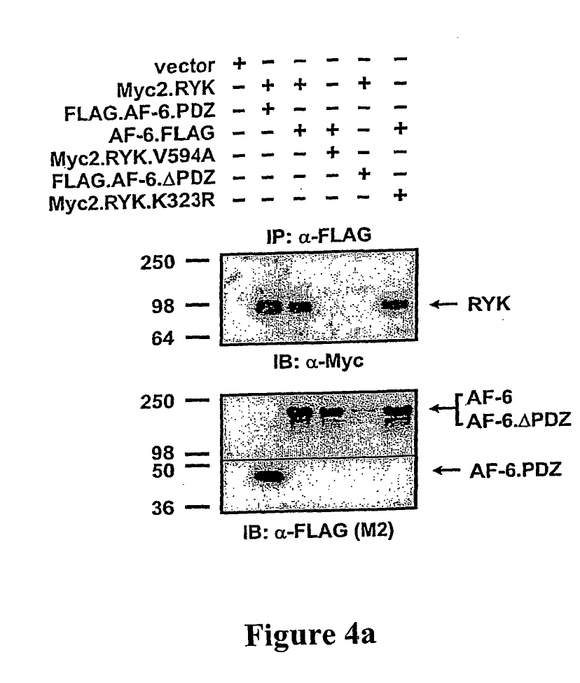

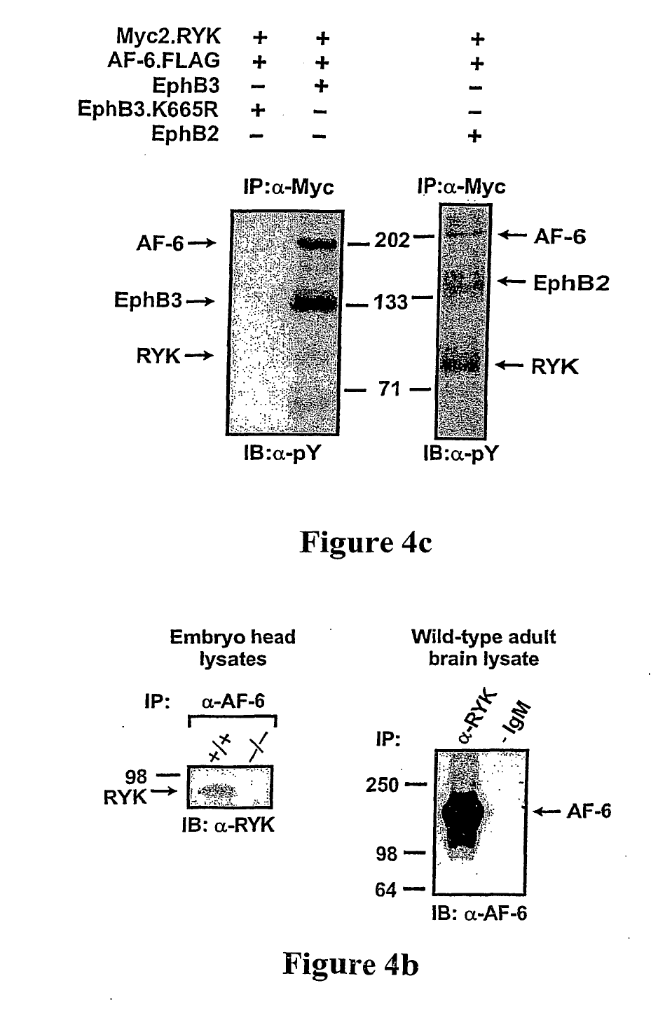

A subset of activated Eph receptors including EphB2, EphB3 and EphA7 bind AF-6 (12, 13), a cell junction-associated target of activated Ras subfamily members (14, 15). Yeast two-hybrid analysis of the mouse RYK cytoplasmic domain identified an interaction with the PDZ domain of AF-6, indicating that this scaffold protein may be a common target of RYK and Eph receptors. Wild-type RYK was efficiently coprecipitated from 293T cells transfected with full-length AF-6, unlike a V594A mutant of RYK involving substitution of the critical C-terminal valine residue (FIG. 4a). Deletion of the single PDZ domain from AF-6 likewise abrogated coprecipitation of RYK. Any residual kinase activity displayed by RYK was unnecessary for interaction with AF-6, as assessed by successful coprecipitation of RYK.K323R (bearing a mutation in PTK subdomain II known to inactivate PTK catalytic activity and to destroy the ability of RYK to activate the ERK MAPK pathway (6)) with full-length AF-6 (FIG. 4a).

In contrast to RYK, Eph receptors B2 and B3 positively regulate AF-6 binding to their C-termini by phosphorylation of AF-6 on tyrosine residues (12, 13). When Myc2.RYK was coexpressed with these Eph receptors and AF-6 in vitro, anti-Myc immunoprecipitates contained tyrosyl-phosphorylated RYK, Eph receptor and AF-6 (FIG. 4b). Anti-AF-6 immunoprecipitates from Ryk^{+/+, but not Ryk}^{-/-,} 14.5 dpc embryonic head lysates were found to contain RYK, indicating that RYK is likely to associate with AF-6, EphB2 and EphB3 in vivo (FIG. 4c; see also FIG. 3d).

The inventors have demonstrated genetically an essential role for the Ryk gene in animal limb and craniofacial development. Furthermore, the biochemical data implicate RYK in signal crosstalk with EphB2 and EphB3, involving Eph receptor-dependent transphosphorylation of RYK on tyrosine residues. Although not intending to limit the present invention to one theory or mode of action, the apparently constitutive association of RYK with these Eph receptors and the AF-6 scaffolding molecule may reflect recruitment of a preassembled complex of Eph receptor effectors to the appropriate subcellular location. Combinatorial interaction of RYK with specific Eph subfamily members suggests the potential for subtle defects in axon guidance and/or angiogenesis in RYK-deficient mice, similar to those phenotypes reported in Derailed-deficient Drosophila strains (8, 9, 16) and EphB2/EphB3-deficient mice (7, 17). Interestingly, craniofacial and limb defects resembling those of RYK-deficient mice are common features of human syndromes associated with changes in gene dosage at 3q22-qter (e.g. 18-25) where RYK and EPHB3 are located (26, 27). These loci now become candidates for involvement in such human craniofacial disorders.

TABLE 1
Viability of Ryk mutant mice (RKO.44 strain)

| Stage^{a} | Ryk^{+/+} | Ryk^{+/-} | Ryk^{-/-} | Total |
|---|---|---|---|---|
| E18.0 | 3 | 12 | 5 | 20 |
| E18.5 | 26 | 33 | 19 | 78 |
| E19.0 | 6 | 10 | 7 | 23 |
| Newborn^{b} | 41 | 89 | 45 | 175 |
| Weaning^{c} | 243 | 410 | 2 | 655 |

^{a}Noon on day of detection of copulation plug defined as 0.5 dpc.
^{b}Genotyped within 10 hours of birth.
^{c}Typically 2-3 weeks of age.

TABLE 2
Limb and craniofacial bone dimensions in neonatal Ryk^{-/-} and littermate mice^{a}

| | Ryk^{+/+,b} | | Ryk^{+/-,c} | | Ryk^{-/-,d} | |
|---|---|---|---|---|---|---|
| | Length | Width | Length | Width | Length | Width |
| Ulna | 3.4 ± 0.3 | 0.38 ± 0.02 | 3.37 ± 0.16 | 0.39 ± 0.02 | 2.54 ± 0.19 (<0.005) | 0.42 ± 0.03 (0.04) |
| Fibula | 3.73 ± 0.25 | 0.25 ± 0.01 | 3.60 ± 0.12 | 0.24 ± 0.01 | 3.05 ± 0.14 (<0.005) | 0.25 ± 0.01 (0.17) |
| Nasal bone | 2.11 ± 0.16 | 2.36 ± 0.09 | 2.27 ± 0.07 | 2.28 ± 0.11 | 1.78 ± 0.14 (<0.005) | 2.32 ± 0.14 (0.60) |
| Mandible | 5.21 ± 0.23 | 1.49 ± 0.16 | 5.28 ± 0.34 | 1.58 ± 0.05 | 4.78 ± 0.40 (<0.018) | 1.43 ± 0.08 (0.005) |
| Interparietal bone | 1.82 ± 0.27 | 4.21 ± 0.26 | 1.94 ± 0.11 | 4.21 ± 0.15 | 1.88 ± 0.13 (0.31) | 4.04 ± 0.23 (0.12) |

^{a}Numbers are mean ± SD in millimetres. Parentheses indicate P value derived from a two tailed t-test comparing Ryk^{+/-} and Ryk^{-/-}.
^{b}N = 7.
^{c}N = 17.
^{d}N = 8.

### BIBLIOGRAPHY

1. Ferguson, M. W. Development 103 Suppl:41-60 (1988).

2. Gorlin et al. Syndromes of the head and neck, (Oxford University Press, New York, 1990).

3. Francis-West et al. Mech. Dev. 75:3-28 (1998).

4. Hovens, C. M. et al. Proc. Natl. Acad. Sci. U.S.A. 89:11818-11822 (1992).

5. Halford et al. J. Biol. Chem. 274:7379-7390 (1999).

6. Katso et al. Mol. Cell Biol. 19:6427-6440 (1999).

7. Orioli et al. EMBO J. 15:6035-6049 (1996).

8. Callahan Nature 376:171-174 (1995).

9. Bonkowsky Nature 402:540-544 (1999).

10. Kamitori Dev. Brain. Res. 114:149-160 (1999).

11. Ciossek Oncogene 9:97-108 (1995).

12. Hock et al. Proc. Natl. Acad. Sci. U.S.A. 95:9779-9784 (1998).

13. Buchert et al. J. Cell. Biol. 144:361-371 (1999).

14. Kuriyama et al. J. Biol. Chem. 271:607-610 (1996).

15. Linnemann et al. J. Biol. Chems. 274:13556-13562 (1999).

16. Simon et al. Mech. Dev. 76:45-55 (1998).

17. Adams et al. Genes Dev. 13:295-306 (1999).

18. Salazar et al. Am. J. Dis. Child. 133:1006-1008 (1979).

19. Fear et al. Arch. Dis. Child. 54:135-138 (1979).

20. Rosenfeld et al. Am. J. Med. Genet. 10:187-192 (1981).

21. Williamson et al. Am. J. Med. Genet. 9:105-111 (1981).

22. Fujita et al. Am. J. Med. Genet. 44:434-436 (1992).

23. Jewett et al. Am. J. Med. Genet. 47:1147-1150 (1993).

24. Ishikiriyama, S. & Goto, M. Am. J. Med. Genet. 52:245 (1994).

25. Fryns, J. P. Clin. Genet. 48:111-112 (1995).

26. Stacker et al. Oncogene 8:1347-1356 (1993).

27. Böhme et al. Oncogene 8:2857-2862 (1993).

28. Hogan et al. Manipulating the mouse embryo: a laboratory manual, (Cold Spring Harbour Laboratory Press, Cold Spring Harbour, N.Y., 1994).

29. Schneider et al. Nature Biotechnol. 17:170-175 (1999).

30. Koblizek et al. Eur. J. Biochem. 244:774-779 (1997).

31. Douillard and Hoffman, Basic Facts about Hybridomas, in Compendium of Immunology Vol. II, ed. by Schwartz (1981);

32. Kohler and Milstein, Nature 256:495-499 (1975); European Journal of Immunology 6:511-519, (1976).

33. Sambrook et al., “Molecular Cloning. A Laboratory Manual”, Cold Spring Harbor Press, 1989.

1. A method for detecting a likelihood for progression of a developmental abnormality in an animal or for diagnosing the genetic or biochemical basis behind a particular developmental abnormality in said animal, said method comprising screening for the presence of a functional RYK molecule or its homologue or a mediator of RYK signalling or a functional nucleic acid molecule encoding said RYK or its signalling mediator or a transcript thereof wherein the absence of a functional RYK or its homologue or its signalling mediator or a mutation in the nucleic acid molecule encoding RYK or its signalling mediator or the presence of an aberration in the RYK transcript or signalling mediator transcript is indicative of or the likelihood of progression to a developmental disorder in said animal.

2. A method according to claim 1 wherein the animal is a mammal.

3. A method according to claim 2 wherein the mammal is a human.

4. A method according to claim 1 wherein the developmental disorder is an aberration in morphogenesis of craniofacial structures.

5. A method according to claim 1 wherein the developmental disorder is a neural condition.

6. A method according to claim 5 wherein the neural condition is an aberration in axon guidance.

7. A method according to claim 5 wherein the aberration results in a failure for axons to cross the midline.

8. A method according to claim 1 wherein the developmental disorder affects angiogenesis or muscle development.

9. A method according to claim 1 wherein a RYK signalling mediator is an Eph receptor.

10. A method according to claim 9 wherein the Eph receptor is selected from EphB3, EphB3, EphA7 and EphB2.

11. A method according to claim 1 wherein the RYK signalling mediator is AF-6.

12. A method for detecting a likelihood for the progression of abnormal craniofacial structures in an animal or for diagnosing the genetic or biochemical basis behind a particular craniofacial abnormality or for detecting neurological conditions, conditions affecting angiogenesis and/or muscle development or maintenance in said animal, said method comprising screening for the presence of a functional RYK molecule or its homologue or a mediator of RYK signalling or a functional nucleic acid molecule encoding said RYK or its signalling mediator or a transcript thereof wherein the absence of a functional RYK or its homologue or its signalling mediator or a mutation in the nucleic acid molecule encoding RYK or its signalling mediator or the presence of an aberration in the RYK transcript or signalling mediator transcript is indicative of or the likelihood of progression to an abnormality in said animal.

13. A method according to claim 12 wherein the animal is a mammal.

14. A method according to claim 13 wherein the mammal is a human.

15. A method according to claim 12 wherein a RYK signalling mediator is an Eph receptor.

16. A method according to claim 15 wherein the Eph receptor is selected from EphB3, EphB3, EphA7 and EphB2.

17. A method according to claim 12 wherein the RYK signalling mediator is AF-6.

18. A method for detecting an abnormal genomic coding sequence for a RYK or coding for a protein having RYK-like properties in an animal subject, said method comprising contacting a genetic sample from said subject with one or more oligonucleotide primers specific for a part of the naturally occurring genomic sequence for said protein or for an abnormal coding sequence for said protein for a time and under conditions sufficient for said oligonucleotides to hybridize to said genomic sequence and then screening for said hybridization.

19. A method according to claim 18 wherein the animal is a mammal.

20. A method according to claim 19 wherein the mammal is a human.

21. A method according to claim 18 further comprising amplifying a genomic sequence.

22. A genetically modified animal comprising the genotype Ryk^{-/-} or Ryk^{+/-}.

23. Use of a genetically modified animal according to claim 22 to screen for a chemical or natural products which block, reverse or otherwise ameliorate the effects of a mutated Ryk phenotype.

24. A genetically modified animal according to claim 22 wherein the animal is a mouse.


SEQUENCE LISTING (9 sequences)

SEQ ID NO: 1 - DNA, 19 bp, primer
gcgttggctacccgtgata

SEQ ID NO: 2 - DNA, 22 bp, primer
caagtaacatgctccccaaaac

SEQ ID NO: 3 - DNA, 20 bp, primer
cagggcagtgcattccatct

SEQ ID NO: 4 - DNA, 18 bp, primer
ttgtggctatgggcatgc

SEQ ID NO: 5 - DNA, 18 bp, primer
gaaatatcactgcacagc

SEQ ID NO: 6 - DNA, 19 bp, primer
catatggctattcagattg

SEQ ID NO: 7 - DNA, 21 bp, primer
tggaccagctgctggaactaa

SEQ ID NO: 8 - DNA, 21 bp, primer
gactgtggccggctgggtgtg

SEQ ID NO: 9 - DNA, 22 bp, primer
gggcgtcgcttggtcggtcatt